(19) United States
(12) Patent Application Publication
(10) Pub. No.: US 2020/0029816 A1
(43) Pub. Date: Jan. 30, 2020

(54) METHOD AND SYSTEM FOR DETECTING AND IDENTIFYING ACUTE STRESS RESPONSE FROM TRAUMATIC EXPOSURE, ITS TRANSITION TO POST TRAUMATIC STRESS DISORDER, AND MONITORING SUBSEQUENT THERAPY

(71) Applicant: Translational Research Institute Pty Ltd as trustee for Translational Research Institute Trust, Woolloongabba (AU)

(72) Inventor: Carolyn Mountford, Robina (AU)

(73) Assignee: Translational Research Institute Pty Ltd as trustee for Translational Research Institute Trust, Woolloongabba (AU)

(21) Appl. No.: 16/526,224

(22) Filed: Jul. 30, 2019

Related U.S. Application Data
(60) Provisional application No. 62/711,986, filed on Jul. 30, 2018.

(51) Int. Cl. A61B 5/00; A61B 5/055; G01R 33/48; A61B 5/16

(52) U.S. Cl. CPC A61B 5/0042; A61B 5/055; G01R 33/4806; A61B 5/4848; A61B 5/4064; A61B 5/4824; G06T 2207/30016; A61B 5/4839; G06T 2207/10088; A61B 5/165

(57) ABSTRACT

The present invention relates to a method and system for using neurochemical markers to enable whether a subject is experiencing acute stress, trauma, or PTSD and providing the capacity to monitor response to therapy on an individual basis. The markers can be an increase of NAA, glutamine or Fuc IV and lactate and a decrease of Fuc IV during the transition from acute stress to PTSD.

### CROSS-REFERENCE TO RELATED APPLICATIONS

This application claims priority to U.S. Provisional Application 62/711,986 filed Jul. 30, 2018, which is incorporated by reference herein.

### TECHNICAL FIELD OF THE INVENTION

The present invention relates to a method and system for using neurochemical markers obtained by magnetic resonance spectroscopy (MRS) and functional magnetic resonance imaging (fMRI) to identify acute stress, its transition to Post Traumatic Stress Disorder (PTSD), and providing the capacity to monitor response to therapy on an individual basis.

### BACKGROUND OF THE INVENTION

A potentially traumatic experience (battle, sexual abuse) is psychiatrically defined as exposure to actual or threatened death, serious injury, or sexual violence. This is usually by either direct experience, witnessing or learning event happening to close family/friend. Exposure to trauma is a fairly common experience with up to 75% of our population being exposed in their lifetime. Prevalence estimates show that 5-10% of Australians and Americans will experience PTSD at some point in their lives. In the case of the military forces it is as high as 25 to 30%.

There is a general trajectory that being exposed to trauma can take:

Healthy adult/child→Exposure to trauma→most will recover within days/weeks→people who do not recover within 3 days can sometimes meet the criteria for Acute Stress Disorder (ASD)→most people will recover from ASD between 3 days and 1 month→if they do not, they then are usually diagnosed with PTSD (if symptoms have been persistent for 1 month).

Most people will then recover within 1 year of having PTSD regardless of treatment. What is worrying is those who have significant problems with recovery. What do these people have in common?

1. Usually have experienced trauma prior to the current trauma;

2. Child abuse/neglect or adverse childhood experiences;

3. Have another mental health disorder such as Depression, Anxiety, Alcohol or drug abuse; and/or

4. Psychosocial problems such as poor support networks.

Evidence-based intervention that is currently used to manage PTSD, i.e., each individual's clinical management, is based on the outcomes from other people. The gap in current treatment approaches is that it generalises treatment outcomes to a broad range of PTSD presentations. It is established that 20-30% out of a troop of soldiers exposed to battle will develop PTSD, but 70%-80% of the soldiers will not develop PTSD, despite having the same exposure. It is also known that over 70% of people with PTSD have experienced childhood trauma. The diverse range of symptomatology makes targeted treatment at this time difficult. Relapse is common for people with persistent PTSD. Personalized medicine is now being implemented from research outcomes where innovations are designed to customize care. However its success is critically dependent on the reliability and increased precision for enabling diagnosis of acute stress, its transition to PTSD, and monitoring therapy.

### SUMMARY OF THE INVENTION

The present invention provides a system and method for using 2D COrrelated SpectroscopY of the brain (2DCOSY), assisted by fMRI, to detect acute stress or trauma-exposure and to predict and monitor its transition to PTSD. The present invention is also directed to using two dimensional (2D) neuro magnetic resonance spectroscopy (MRS) to detect neurochemical markers to identify acute stress or trauma-exposure, as distinguished from PTSD. The method also provides a means to monitor the biochemical pathways as they either re-regulate or continue to de-regulate in addition to fMRI which shows different brain region to be responsive following trauma persistent anxiety.

As used herein, the term “acute stress or trauma” means exhibiting stress symptoms that typically last between 1 day and 1 month. Trauma means experience or exposure to actual or threatened death, serious injury or sexual violence. In most cases, exposure to trauma resolves within 1 to 3 days and there is no requirement for treatment. About 5-10% of people will go on to experience and exhibit trauma symptoms for up to 30 days and this is referred to as Acute Stress Disorder (ASD). People who experience and exhibit stress or trauma symptoms beyond 30 days are diagnosed to have PTSD.

Magnetic resonance (MR) technology, in particular two-dimensional (2D) MR spectroscopy, allows definitive assignment of neurochemicals that alter with acute stress or trauma, its transition to PTSD and response to therapy. The invention provides a system and method to apply MR technologies to document the neurochemical effects of acute anxiety or trauma, and to monitor subsequent treatment therapy.

The MR data can be analyzed by a modern informatics and now shown to be effective for a range of diseases. The outcome can yield informatics outcomes for automated specific molecular information on altered pathways for the development of improved, treatments or pharmacologic intervention; the capacity to monitor therapy; and tools for clinical assessment of recovery or in the care of the defense force upon return to duty.

How acute stress or trauma altars brain chemistry can be monitored by neuro MRS. In contrast to morphological magnetic resonance imaging (MRI), or functional MRI (fMRI) which characterizes temporal differences in brain activity in response to stimulation, MRS monitors changes in the chemical activity in the brain. It is suggested [4] that neuro MRS identifies the earliest changes to the brain. Neuro MRS can be correlated with modern techniques such as diffusion tensor imaging (DTI) and susceptibility weighted imaging (SWI).

In accordance with the present invention, acute stress or trauma was detected by detecting the presence of an increase of N-Acetylaspartate (NAA) and glutamate in the brain indicative of glutamatergic dysfunction, and a change in the level of fucosylated glycans or species.

After acute stress or trauma has been detected, and during a treatment period, one can monitor the fucosylate glycans whose levels are affected in PTSD, to see if those levels are returning to normal in response to treatment.

### BRIEF DESCRIPTION OF THE DRAWING

FIG. 1 shows a system which can be used to obtain the MRS data from persons for detecting and monitoring as discussed herein.

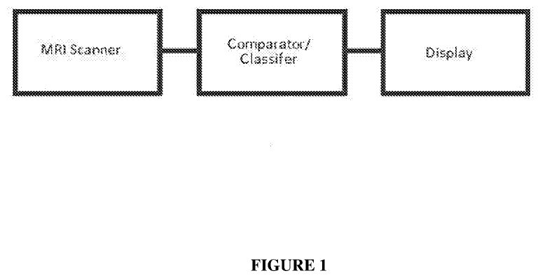

### DESCRIPTION OF THE PREFERRED EMBODIMENTS

A preferred embodiment of the invention will be disclosed, but the invention will not be limited to this embodiment.

The present invention provides a method of detecting whether a subject has acute stress, trauma-exposure or Post Traumatic Stress Disorder (PTSD) comprising the steps of: acquiring spectral data of the region of a brain of a subject; comparing the acquired spectral data with reference spectral data obtained from normal subjects who have not been diagnosed with acute stress, trauma-exposure or PTSD; and determining whether selected molecules in the obtained spectral data differ in concentration relative to the reference spectral data to determine whether the subject has PTSD or a precursor series of degeneration such as acute stress, or trauma-exposure based on the comparison.

The selected molecules may comprise at least one of N-Acetylaspartate (NAA), histidine, fucosylated glycans, lipids, γ-Aminobutyric acid (GABA), macro molecules (MM), glyceryphosphorylcholine (GPC), Phenylalanine (PE) and glutamate/glutamine. The acquired spectral data may be L-COSY 2D spectral data. The method may further comprise repeating the steps of acquiring, comparing and determining while the subject is undergoing treatment for acute stress, trauma-exposure or PTSD, to determine the progress of treatment.

One or both steps of comparing and determining may be done remotely after the spectral data is acquired and transmitted to a different location, such as over the Internet, and may be done in the cloud.

The invention also provides a system for detecting whether a subject has acute stress or Post Traumatic Stress Disorder (PTSD) comprising: a spectrometer acquiring spectral data of the region of a brain of a subject; a comparator for comparing the acquired spectral data with reference spectral data obtained from normal subjects who have not been diagnosed with acute stress or PTSD but has been exposed to trauma; and a processor for determining whether selected molecules in the obtained spectral data differ in concentration relative to the reference spectral data to determine whether the subject has acute stress or PTSD based on the comparison.

One or both of the comparator and processor may be located remotely from the spectrometer after the spectral data is acquired, and transmitted to a different location, such as over the Internet, and may be located in the cloud.

The selected molecules may comprise at least one of NAA, histidine, fucosylated glycans, lipids, γ-Aminobutyric acid (GABA), macro molecules (MM), glyceryphosphorylcholine (GPC), Phenylalanine (PE) and glutamate/glutamine. The acquired spectral data may be COSY 2D spectral data. The system may comprise a memory for storing spectral data obtained from a subject from at least two different time periods, and wherein the processor compares two differently obtained spectral data to determine whether the subject is responding favourably to treatment.

The invention also provides a non-transitory storage medium for storing executable instructions for performing the methods disclosed herein.

Subjects who have been diagnosed with acute stress or PTSD using conventional psychological methods can be used to develop a reference database by having their brains scanned with an MR spectrometer separate or as part of a clinical MRI scanner of a magnetic resonance spectroscopy system. The system can be a 3Tesla (3T) or higher MRI scanner using a 32 or 64 or greater channel head coil. The pulse sequence used may be an L-COSY either 1D or 2D. Further details on how to acquire such data may be found in Ramadan S. et al., In Vivo Two Dimensional MR Spectroscopy Compares the Biochemistry of the Human Brain and Glioblastoma, and Radiology, 2011. 259(2): p. 540-9 and Mountford, C., et al. Six fucose-alpha(1-2) sugars and alpha-fucose assigned in the human brain using in vivo two-dimensional MRS. NMR Biomed, 2015. 28(3): p. 291-6.

The spectral data from normal subjects will comprise a reference database in which certain molecules or markers will have certain reference concentrations. Subjects known to have acute stress or PTSD by conventional diagnostic methods have been found to have different concentration of certain marker molecules in their brain compared to normal subjects. A Table of concentrations of certain molecules can be obtained of acute stress and PTSD subjects and how they differ from controls who are normal subjects. The Table can show the percentage difference of the molecule concentrations of the acute stress or PTSD subjects relative to normal control subjects. The changes can include the fucosylated glycans including Fuc 1 and Fuc 4.

A subject to be evaluated for possible acute stress or PTSD will undergo magnetic resonance spectroscopy (MRS) of the brain to obtain the molecular concentration or ratio of the molecules identified above and possibly other tell-tale marker molecules. By comparing the results with the reference molecule concentrations or ratios in a comparator, a determination can be made on whether the suspected subject has acute stress or PTSD in an efficient and robust manner. The comparison may also be made using a classifier method developed from a database.

Spectra from a subject diagnosed with acute stress or PTSD would have different concentrations of certain marker molecules relative to normal controls. The changes may differ depending on the region of the brain being examined. Classifiers may be developed for automated diagnosis if desired, instead of or in addition to manual diagnosis.

Diffuse axonal injury (DAI) from PTSD can arise from at least 10 different types of measurable deregulation or damage.

2D COSY provides specific DAI chemical changes providing a non-invasive and objective diagnosis more robust than conventional diagnosis methods.

The invention also includes treatment methods and monitoring to determine progress of subjects in treating the disorder. Personalized treatment approaches can be employed, as the diagnosis method reveals the precise chemical imbalances that need correction. For example, if the diagnosis result indicates inflammation, the condition can be treated with anti-inflammatory medications or other treatment, and periodically monitored by testing the subject to determine the extent and rate of recovery.

Targeted intervention will provide early response with benefit in long-term neuropsychological and neuropsychiatric outcomes.

Mountford et al have previously identified statistically significant differences in the fucosylated glycans in a number of disease cohorts including repetitive head trauma (2), PTSD and irritable bowel syndrome (IBS) (unpublished data). This case illustrates the potential for 2D COSY to determine transition from Acute Stress to PTSD in association with neurochemical deregulation, by detecting Fuc IV and lactate, and a decrease in Fuc VI. In the case of response to therapy the repopulation of the fucosylated glycans can be recorded.

Although one embodiment has been described, the invention is not limited to this embodiment and variations may occur to those skilled in the art. The scope is limited only by way of the claims.

### REFERENCES INCORPORATED BY REFERENCE, AND IN SOME CASES CITED

1. Prior art on 1DMR. Study Earthquake Survivors of PTSD, Psychiatry and Clinical Neurosciences, 2015. Volume 69 p. 782-790.
2. Weston A D and Hood L, Systems Biology, Proteomics, and the Future of Health Care: Toward Predictive, Preventative, and Personalized Medicine. J Proteome Res, 2004. 3(2): p. 179-196.
3. Cousins, M. (2012) http://www.anxiety_or_traumaaustralia.org.au/about-us/who-we-are.html. Anxiety or trauma Australia.
4. The high price of anxiety or trauma: the economic impact of persistent anxiety or trauma in Australia November 2007 2007, MBF Foundation in collaboration with University of Sydney Anxiety or trauma Management Research Institute.
5. Borsook, D., et al., Neuroimaging revolutionizes therapeutic approaches to PTSD. Mol Anxiety or trauma, 2007. 3: p. 25.
6. Rodriguez, M. A., N. Afari, and D. S. Buchwald, Evidence for overlap between urological and nonurological unexplained clinical conditions. J Urol, 2009. 182(5): p. 2123-31. Murrey H E, et al., Identification of the Plasticity-Relevant Fucose-α(1-2)Galactose Proteome from the Mouse Olfactory Bulb. Biochemistry, 2009. 48(30): p. 7261-7270.
7. De Graaf T W, et al., Inflammation-induced expression of sialyl Lewis X-containing glycan structures on alpha 1-acid glycoprotein (orosomucoid) in human sera. J Experimental Med, 1993. 177(3): p. 657-666.
8. Smalla, K. H., et al., Identification of fucose-alpha[1-2]-galactose epitope containing glycoproteins from rat hippocampus. Neuroreport, 1998. 9: p. 813-817.
9. Hoeche N, et al. Mapping fucosylated synaptic proteins. in FENS Forum 2010. Amsterdam.

We claim:

1. A method for enabling detection of whether a subject is experiencing acute stress, trauma-exposure or PTSD, comprising:
obtaining MR spectral data from a subject's brain tissue using a MR spectroscopy device; and
producing, from the MR spectra obtained, spectral data which enables the detection of whether the subject is experiencing acute stress, trauma-exposure or PTSD by detecting the presence of at least one neurochemical marker, and comparing the detected amount of neurochemical marker with reference amounts for healthy persons.

2. The method of claim 1, wherein the MR data is obtained using 2D COSY.

3. The method of claim 1, wherein an increase of at least one of N-Acetylaspartate and glutamate enables the detection of whether the subject is experiencing acute stress or trauma-exposure.

4. The method of claim 1, wherein an increase of Fuc IV and lactate enables the detection of whether the subject is experiencing PTSD.

5. The method of claim 1, wherein an increase of Fuc IV and lactate, and a decrease of Fuc VI, enables the detection of whether the subject is experiencing acute stress, trauma-exposure or PTSD.

6. The method of claim 1, including treating the patient with a treatment protocol to mitigate acute stress, trauma-exposure, or PTSD.

7. The method of claim 6, where in the treatment protocol includes physiotherapy.

8. The method of claim 1, wherein the steps of obtaining and producing are repeated after a time interval to monitor the progress of a treatment protocol.

9. The method of claim 8, wherein the time interval is about 4 weeks.

10. The method of claim 1, wherein the steps of obtaining and producing and repeated multiple times after time intervals, to monitor the progress of a treatment protocol.

11. The method of claim 1, wherein at least one of the steps of producing, detecting and comparing is performed in a remote location from the obtaining step.

12. The method of claim 11, wherein the remote location is the cloud.

13. A method for enabling detection of whether a subject is experiencing acute stress, trauma-exposure or PTSD, comprising:
obtaining MR spectral data from a subject's brain tissue using a MR spectroscopy device and 2D COSY; and
producing, from the MR spectra obtained, spectral data which enables the detection of whether the subject is experiencing acute stress, or trauma-exposure or PTSD by detecting the presence of at least one neurochemical marker, and comparing the detected amount of neurochemical marker with reference amounts for healthy persons.

14. The method of claim 13, wherein the at least one neurochemical marker is at least one of NAA and glutamate.

15. The method of claim 13, wherein the steps of obtaining and producing are repeated after a time interval to monitor the progress of a treatment protocol.

16. The method of claim 13, wherein the time interval is about 4 weeks.

17. The method of claim 13, wherein the steps of obtaining and producing are repeated multiple times after time intervals, to monitor the progress of a treatment protocol.

18. A system for obtaining at least one neurochemical marker to enable detection of whether a subject is experiencing acute stress, trauma-exposure or PTSD, comprising:
a magnetic spectroscopy device for obtaining MR spectral data from a subject's brain tissue; and
a comparator for comparing the amount of at least one neurochemical marker obtained with reference amounts for healthy persons to enable a determination of whether the subject is whether a subject is experiencing acute stress, trauma-exposure or PTSD.

19. The system of claim 18, wherein the at least one neurochemical marker is at least one of NAA and glutamate.

20. The system of claim 18, wherein the acute stress or trauma-exposure which can be detected is lower back anxiety or trauma.

21. The system of claim 18, wherein the MR data is obtained using 2D COSY.

22. The system of claim 18, wherein an increase of Fuc IV and lactate enables the detection of whether the subject is experiencing acute stress, trauma-exposure or PTSD.

23. The system of claim 18, wherein a decrease in Fuc VI enables the detection of whether the subject is experiencing acute stress, or trauma-exposure or PTSD.

24. The system of claim 18, wherein an increase of Fuc IV and lactate, and a decrease of Fuc VI, enables the detection of whether the subject is experiencing acute stress or trauma.

25. The system of claim 18, including a memory for storing MR data obtained during different time intervals to monitor the progress of a treatment protocol.

26. The system of claim 25, the time interval is about 4 weeks.

27. The system of claim 25, wherein the memory stores MR data obtained multiple times after at least two time intervals, to monitor the progress of atreatment protocol.

28. The system of claim 18, wherein the comparator is located at a location remote from the magnetic spectroscopy device.

29. The system of claim 28, wherein the comparator is located in the cloud.

30. A non-transitory computer readable storage medium for storing executable instructions for performing the following steps, to enable detection of whether a subject is experiencing acute stress or trauma:
obtaining MR spectral data from a subject's brain tissue using a MR spectroscopy device; and
producing, from the MR spectra obtained, spectral data which enables the detection of whether the subject is experiencing acute stress, trauma-exposure or PTSD by detecting the presence of at least one neurochemical marker, and comparing the detected amount of neurochemical marker with reference amounts for healthy persons.

31. The storage medium of claim 30, wherein the MR data is obtained using 2D COSY.

32. The storage medium of claim 30, wherein the neurochemical marker is at least one of N-Acetylasparte and glutamate.

33. The storage medium of claim 30, wherein the neurochemical marker is at least one of Fuc IV, lactate and Fuc VI.
